(19) United States
(12) Patent Application Publication
(10) Pub. No.: US 2012/0270945 A1
(43) Pub. Date: Oct. 25, 2012

(54) METHOD FOR MODULATING CYTOKINE ACTIVITY

(75) Inventor: Ryuji UENO, Montgomery, MD (US)

(73) Assignee: SUCAMPO AG, Zug (CH)

(21) Appl. No.: 13/450,071

(22) Filed: Apr. 18, 2012

Related U.S. Application Data
(60) Provisional application No. 61/476,992, filed on Apr. 19, 2011.
(60) Provisional application No. 61/489,516, filed on May 24, 2011.
(60) Provisional application No. 61/537,305, filed on Sep. 21, 2011.
(60) Provisional application No. 61/548,458, filed on Oct. 18, 2011.

(51) Int. Cl. A61K 31/20; A61P 1/00; A61P 37/02

(52) U.S. Cl. 514/559

(57) ABSTRACT

The present invention relates to a method for modulating cytokine activity, immunomodulation or treating esophagitis comprising an administration of a fatty acid derivative to a mammalian subject. The present invention also relates to a composition for modulating cytokine activity, immunomodulation or treating esophagitis comprising a fatty acid derivative.

### TECHNICAL FIELD

The present invention relates to a method for modulating cytokine activity. The present invention also relates to a method for immunomodulation.

### BACKGROUND

Cytokines and chemokines are proteins secreted from cells upon activation, which regulate the survival, proliferation, differentiation and function of a variety of cells within the living body. They are important in cellular communication, and in regulating responses to homeostasis or biophylaxis. Cytokines are the general category of signaling molecules produced by various types of cells such as T cells that direct the immune response, while chemokines are a special type of cytokine that direct the migration of white blood cells to infected or damaged tissues. A cytokine and a chemokine both use chemical signals to induce changes in other cells, but the latter are specialized to cause cell movement.

Cytokines include, for example, interleukin (IL) including over 30 type such as IL-1α, IL-1β, IL-2, -3, -4, -5, -6, -7, -8, -9, -10, -11 to -37; interferon (IFN) such as IFN-α, IFN-β and IFN-γ; tumor necrosis factor (TNF) such as TNF-α and TNF-β; transforming growth factor (TGF) such as TGF-α and TGF-β; colony stimulating factor (CSF) such as granulocyte-colony-stimulating factor (G-CSF), granulocyte-macrophage-colony-stimulating factor (GM-CSF), macrophage-colony Stimulating factor (M-CSF), erythropoietin (EPO), stem cell factor (SCF) and monocyte chemotactic and activating factor (MCAF); growth factor (GF) such as epidermal growth factor (EGF), fibroblast growth factor (FGF), insulin like growth factor (IGF), nerve growth factor (NGF), Brain-derived neurotrophic factor (BDNF), platelet derived growth factor (PDGF), vascular endothelial growth factor (VEGF), hepatocyte growth factor (HGF), keratinocyte growth factor (KGF), thrombopoietin (TPO), and bone morphogenic protein (BMP); and other polypeptide factors including LIF, kit ligand (KL), MPO (Myeloperoxidase) and CRP (C-reactive protein), COX (Cyclooxygenase) such as COX-1, COX-2 and COX-3, NOS (Nitric oxide synthase) such as NOS-1, NOS-2 and NOS-3, SOCS (suppressor of cytokine signaling) such as CIS, SOCS-1, -2, -3, -4, -5, -6 and -7, and so on.

There are two major classes of chemokines, CXC and CC. The CXC chemokines, such as neutrophil-activating protein-2 (NAP-2) and melanoma growth stimulatory activity protein (MGSA) are chemotactic primarily for neutrophils and T lymphocytes, whereas the CC chemokines, such as RANTES. Macrophage inflammatory Protein (PUP) including MIP-1α and MIP-1β, keratinocyte-derived chemokine (KC), the monocyte chemotactic proteins (MCP-1, MCP-2, MCP-3, MCP-4, and MCP-5) and the eotaxins (-1 and -2) are chemotactic for, among other cell types, macrophages, T lymphocytes, eosinophils, neutrophils, dendritic cells, and basophils. There also exist the chemokines lymphotactin-1, lymphotactin-2 (both C chemokines), and fractalkine (a CX3C chemokine) that do not fall into either of the major chemokine subfamilies.

While the activation of these signaling pathways is becoming better understood, little is known of the regulation of these pathways, including employment of negative or positive feedback loops. This is important since once a cell has begun to respond to a stimulus, it is critical that the intensity and duration of the response is regulated and that signal transduction is switched off. It is likewise desirable to increase the intensity of a response systemically or even locally as the situation requires.

Fatty acid derivatives are members of class of organic carboxylic acids, which are contained in tissues or organs of human or other mammals, and exhibit a wide range of physiological activity. Some fatty acid derivatives found in nature generally have a prostanoic acid skeleton as shown in the formula (A):

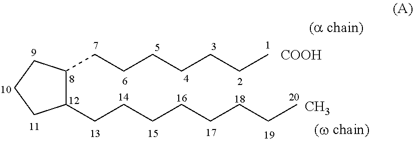

On the other hand, some of synthetic prostaglandin (PG) analogues have modified skeletons. The primary PGs are classified into PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-OH

Subscript 2: 5,6- and 13,14-diunsaturated-15-OH

Subscript 3: 5,6-, 13,14-, and 17,18-triunsaturated-15-OH.

Further, the PGFs are classified, according to the configuration of the hydroxyl group at the 9-position, into α type (the hydroxyl group is of an α-configuration) and β type (the hydroxyl group is of a β-configuration).

PGs are known to have various pharmacological and physiological activities, for example, vasodilatation, inducing of inflammation, platelet aggregation, stimulating uterine muscle, stimulating intestinal muscle, anti-ulcer effect and the like.

Prostones, having an oxo group at position 15 of prostanoic acid skeleton (15-keto type) and having a single bond between positions 13 and 14 and an oxo group at position 15 (13,14-dihydro-15-keto type), are fatty acid derivatives known as substances naturally produced by enzymatic actions during metabolism of the primary PGs and have some therapeutic effect. Prostones have been disclosed in U.S. Pat. Nos. 5,073,569, 5,534,547, 5,225,439, 5,166,174, 5,428,062 5,380,709 5,886,034 6,265,440, 5,106,869, 5,221,763, 5,591,887, 5,770,759 and 5,739,161, the contents of these references are herein incorporated by reference.

However it is not known how fatty acid derivatives act on cytokine activity and its expression.

### DISCLOSURE OF THE INVENTION

The present invention relates to a method for modulating cytokine activity in a mammalian subject, which comprises administering to the subject in need thereof an effective amount of a fatty acid derivative represented by The formula (I):

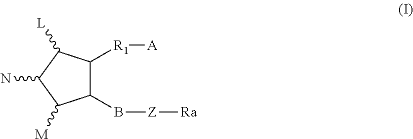

wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —C≡C—CH₂— or —CH₂—C≡C—;

Z is

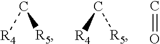

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

The present invention also relates to a method for immunomodulation in a mammalian subject, which comprises administering to the subject in need thereof an effective amount of the fatty acid derivative represented by the formula (I).

The present invention further relates to a method for treating esophagitis in a mammalian subject, which comprises administering to the subject in need thereof an effective amount of the fatty acid derivative represented by the formula (I).

The present invention further relates to a pharmaceutical composition or a composition for modulating cytokine activity, immunomodulation or treating esophagitis comprising an effective amount of the fatty acid derivative represented by the formula (I).

The present invention further relates to use of the fatty acid derivative represented by the formula (I) for the manufacture of a medicament for modulating cytokine activity, immunomodulation or treating esophagitis.

The present invention further relates to use of the fatty acid derivative represented by the formula (I) in modulating cytokine activity, immunomodulation or treating esophagitis.

In one embodiment, the modulation of cytokine activity or the immunomodulation provided by the present invention is useful for treating cytokine-mediated disease or conditions with benefit from immunomodulation.

### BRIEF DESCRIPTION OF DRAWINGS

FIG. 1 shows effects of Compound B on expression of SOCS-1 gene.

### DETAILED DESCRIPTION OF THE INVENTION

The nomenclature of the fatty acid derivative used herein is based on the numbering system of the prostanoic acid represented in the above formula (A).

The formula (A) shows a basic skeleton of the C-20 fatty acid derivative, but the present invention is not limited to those having the same number of carbon atoms. In the formula (A), the numbering of the carbon atoms which constitute the basic skeleton of the fatty acid derivatives starts at the carboxylic acid (numbered 1), and carbon atoms in the α-chain are numbered 2 to 7 towards the five-membered ring, those in the ring are 8 to 12, and those in the ω-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of carboxy group (0-1). Similarly, when the number of carbon atoms is decreased in the ω-chain, the number is deleted in the order starting from position 20; and when the number of carbon atoms is increased in the ω-chain, the carbon atoms at the position or later are named as a substituent at position 20. Stereochemistry of the compounds is the same as that of the above formula (A) unless otherwise specified.

In general, each of PGD, PGE and PGF represents a fatty acid derivative having hydroxy groups at positions 9 and/or 11, but in the present specification they also include those having substituents other than the hydroxy groups at positions 9 and/or 11. Such compounds are referred to as 9-deoxy-9-substituted-fatty acid derivatives or 11-deoxy-1′-substituted-fatty acid derivatives. A fatty acid derivative having hydrogen in place of the hydroxy group is simply named as 9- or 11-deoxy-fatty acid derivative.

As stated above, the nomenclature of a fatty acid derivative is based on the prostanoic acid skeleton. In the case the compound has similar partial structure as the primary PG, the abbreviation of “PG” may be used. Thus, a fatty acid derivative whose α-chain is extended by two carbon atoms, that is, having 9 carbon atoms in the α-chain is named as 2-decarboxy-2-(2-carboxyethyl)-PG compound. Similarly, a fatty acid derivative having 11 carbon atoms in the α-chain is named as 2-decarboxy-2-(4-carboxybutyl)-PG compound. Further, a fatty acid derivative whose ω-chain is extended by two carbon atoms, that is, having 10 carbon atoms in the ω-chain is named as 20-ethyl-PG compound. These compounds, however, may also be named according to the IUPAC nomenclatures.

Examples of the analogues including substitution compounds or derivatives of the above described fatty acid derivative include a fatty acid derivative whose carboxy group at the end of the alpha chain is esterified; a fatty acid derivative whose α chain is extended, a physiologically acceptable salt thereof, a fatty acid derivative having a double bond between positions 2 and 3 or a triple bond between positions 5 and 6; a fatty acid derivative having substituent(s) on carbon atom(s) at position(s) 3, 5, 6, 16, 17, 18, 19 and/or 20; and a tatty acid derivative having a lower alkyl or a hydroxy (lower) alkyl group at position 9 and/or 11 in place of the hydroxy group.

According to the present invention, preferred substituents on the carbon atom at position(s) 3, 17, 18 and/or 19 include alkyl having 1-4 carbon atoms, especially methyl and ethyl. Preferred substituents on the carbon atom at position 16 include lower alkyls such as methyl and ethyl, hydroxy, halogen atom such as chlorine and fluorine, and aryloxy such as trifluoromethylphenoxy. Preferred substituents on the carbon atom at position 17 include lower alkyl such as methyl and ethyl, hydroxy, halogen atom such as chlorine and fluorine, and aryloxy such as trifluoromethylphenoxy. Preferred substituents on the carbon atom at position 20 include saturated or unsaturated lower alkyl such as C₁₋₄ alkyl, lower alkoxy such as C₁₋₄ alkoxy, and lower alkoxy alkyl such as C₁₋₄ alkoxy-C₁₋₄ alkyl. Preferred substituents on the carbon atom at position 5 include halogen atoms such as chlorine and fluorine. Preferred substituents on the carbon atom at position 6 include an oxo group forming a carbonyl group. Stereochemistry of PGs having hydroxy, lower alkyl or hydroxy(lower)alkyl substituent on the carbon atom at positions 9 and 11 may be α, β or a mixture thereof.

Further, the above described analogues or derivatives may have a ω chain shorter than that of the primary PGs and a substituent such as alkoxy, cycloalkyl, cycloalkyloxy, phenoxy and phenyl at the end of the truncated ω-chain.

A fatty acid derivative used in the present Invention is represented by the formula (I):

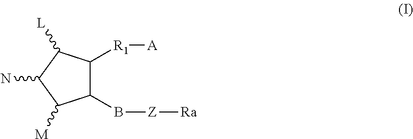

wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;

A is —CH₃, or —CH₂OH, —COCH₂OH, —COOH or a functional 1.5 derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

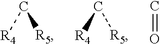

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

A preferred compound used in the present invention is represented by the formula (II):

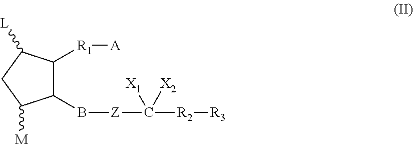

wherein L and M are hydrogen atom, hydroxy, halogen, lower alkyl, hydroxy(lower)alkyl, lower alkanoyloxy or oxo, wherein at least one of L and M is a group other than hydrogen, and the five-membered ring may have one or more double bonds;

A is —CH₃, or —CH₂CH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

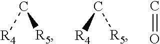

or single bond

wherein R₄ and R₅ are hydrogen, hydroxy, halogen, lower alkyl, lower alkoxy or hydroxy(lower)alkyl, wherein

R₄ and R₅ are not hydroxy and lower alkoxy at the same time;

X₁ and X₂ are hydrogen, lower alkyl, or halogen;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur;

R₂ is a single bond or lower alkylene; and

R₃ is lower alkyl, lower alkoxy, lower alkanoyloxy, cyclo(lower)alkyl, cyclo(lower)alkyloxy, aryl, aryloxy, heterocyclic group or heterocyclic-oxy group, and at least one of carbon atom in the aliphatic hydrocarbon is optionally substituted by oxygen, nitrogen or sulfur.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, isopropylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholine, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydrogen, hydroxy and oxo, and especially, L and M are both hydroxy, or L is oxo and M is hydrogen or hydroxy.

Preferred example of A is —COOH, its pharmaceutically acceptable salt, ester or amide thereof.

Preferred example of X₁ and X₂ are both being halogen atoms, and more preferably, fluorine atoms, so called 16,16-difluoro type.

Preferred R₁ is a hydrocarbon residue containing 1-10 carbon atoms, preferably 6-10 carbon atoms. Further, at least one carbon atom in the aliphatic hydrocarbon is optionally substituted by oxygen, nitrogen or sulfur. Examples of R₁ include, for example, the following groups;

—CH₂—CH₂—CH₂—CH₂—CH₂—CH₂—,

—CH₂—CH═CH—CH₂—CH₂—CH₂—,

—CH₂—CH₂—CH₂—CH₂—CH═CH—,

—CH₂—C≡C—CH₂—CH₂—CH₂—,

—CH₂—CH₂—CH₂—CH₂—O—CH₂—,

—CH₂—CH═CH—CH₂—O—CH₂—,

—CH₂—C≡C—CH₂—O—CH₂—,

—CH₂—CH₂—CH₂—CH₂—CH₂—CH₂—CH₂—,

—CH₂—CH═CH—CH₂—CH₂—CH₂—CH₂—,

—CH₂—CH₂—CH₂—CH₂—CH₂—CH═CH—,

—CH₂—C≡C—CH₂—CH₂—CH₂—CH₂—,

—CH₂—CH₂—CH₂—CH₂—CH₂—CH(CH₃)—CH₂—,

—CH₂—CH₂—CH₂—CH₂—CH(CH₃)—CH₂—,

—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH—,

—CH₂—C═C—CH₂—CH₂—CH₂—CH₂—CH₂—, and

—CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains having one carbon atom. Further, at least one carbon atom in the aliphatic hydrocarbon is optionally substituted by oxygen, nitrogen or sulfur.

Examples of the compounds of the formula (I) or (II) include compounds of the formula (I) wherein Ra is substituted by halogen and/or Z is C═O;

compounds of the formula (II) wherein one of X₁ and X₂ is substituted by halogen and/or Z is C═O;
compounds of the formula (II) wherein L is ═O or —OH, M is H or OH, A is COOH or a functional derivative thereof, B is —CH₂—CH₂—, Z is C═O, X₁ is halogen (e.g. X₁ is Cl, Br, I or F) or hydrogen, X₂ is halogen (e.g. X₂ is Cl, Br, I or F) or hydrogen, R₁ is a saturated or unsaturated bivalent straight C₆ aliphatic hydrocarbon residue, R₂ is a single bond, and R₃ is straight or branched lower alkyl (e.g. C₄₋₆ alkyl) optionally substituted by oxygen, nitrogen or sulfur;
compounds of the formula (II) wherein L is ═O, M is OH, A is COOH or a functional derivative thereof, B is —CH₂—CH₂—, Z is C═O, X₁ is halogen (e.g. X₁ is Cl, Br, I or F) or hydrogen, X₂ is halogen (e.g. X₂ is Cl, Br, I or F) or hydrogen, R₁ is a saturated or unsaturated bivalent straight C₆ aliphatic hydrocarbon residue, R₂ is a single bond, and R₃ is straight or branched lower alkyl optionally substituted by oxygen, nitrogen or sulfur;
compounds of the formula (II) wherein L is ═O, M is OH, A is COOH or a functional derivative thereof, B is —CH₂—CH₂—, Z is C═O, X₁ and X₂ are halogen atoms (e.g. X₁ and X₂ are Cl, Br, I or F), R₁ is a saturated or unsaturated bivalent straight C₆ aliphatic hydrocarbon residue, R₂ is a single bond, and R₃ is straight or branched lower alkyl (e.g. C₄ alkyl or C₅ alkyl);
compounds of the formula (II) wherein L is ═O, M is OH, A is COOH or a functional derivative thereof, B is —CH₂—CH₂—, Z is C═O, X₁ and X₂ are fluorine atoms, R₁ is a saturated or unsaturated bivalent straight C₆ aliphatic hydrocarbon residue, R₂ is a single bond, and R₃ is straight or branched lower alkyl (e.g. C₄ alkyl or C₅ alkyl); and
compounds of the formula (II) wherein L is ═O, M is H or OH, A is COOH or a functional derivative thereof, B is —CH₂—CH₂—, Z is C═O, X₁ and X₂ are halogen atoms (e.g. X₁ and X₂ are Cl, Br, I or F), R₁ is a saturated or unsaturated bivalent straight C₆ aliphatic hydrocarbon residue, R₂ is a single bond, and R₃ is —CH₂—CH₂—CH₂—CH₃ or —CH₂—CH(CH₃)—CH₂—CH₃. The tautomeric isomers of the above-described examples of the compounds of the formula (I) or (II) are also used for the present invention.

Example of the preferred embodiment is a (−)-7-[(2R,4aR,5R,7aR)-2-(1,1-difluoropentyl)-2-hydroxy-6-oxooctahydrocyclopenta[b]pyran-5-yl]heptanoic acid (lubiprostone), (−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid (cobiprostone), (+)-isopropyl (Z)-7-[(1R,2R,3R,5S)-3,5-dihydroxy-2-(3-oxodecyl)cyclopentyl]hept-5-enoate (isopropyl unoprostone) and (−)-7-[(1R,2R)-2-(4,4-difluoro-3-oxooctyl)-5-oxocyclopentyl]heptanoic acid, its tautomeric isomers thereof or its functional derivative thereof.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

In the present invention, the fatty acid derivative which is dihydro between 13 and 14, and keto(═O) at 15 position may be in the keto-hemiacetal equilibrium by formation of a hemiacetal between hydroxy at position 11 and keto at position 15.

For example, it has been revealed that when both of X₁ and X₂ are halogen atoms, especially, fluorine atoms, the compound contains a tautomeric isomer, bicyclic compound.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the present invention includes both isomers.

Further, the fatty acid derivatives used in the invention include the bicyclic compound and analogs or derivatives thereof.

The bicyclic compound is represented by the formula (III)

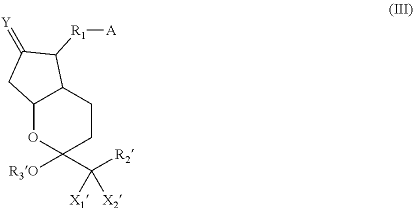

wherein, A is —CH₃, or —CH₂OH, —COCH₂OH, —COOH or a functional derivative thereof;

X₁′ and X₂′ are hydrogen, lower alkyl, or halogen;

Y is

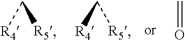

wherein R₄′ and R₅′ are hydrogen, hydroxy, halogen, lower alkyl, lower alkoxy or hydroxy(lower)alkyl, wherein R₄′ and R₅′ are not hydroxy and lower alkoxy at the same time.

R₁ is a saturated or unsaturated divalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

R₂′ is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

R₃′ is hydrogen, lower alkyl, cyclo(lower)alkyl, aryl or heterocyclic group.

Furthermore, while the compounds used in the invention may be represented by a formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324, 5,739,161 and 6,242,485 (these cited references are herein incorporated by reference).

The mammalian subject may be any mammalian subject including a human. The compound may be applied systemically or topically. Usually, the compound may be administered by oral administration, intranasal administration, inhalational administration, intravenous injection (including infusion), subcutaneous injection, ocular topical administration, intra rectal administration, intra vaginal administration, transdermal administration and the like.

The dose may vary depending on the strain of the animal, age, body weight, symptom to be treated, desired therapeutic effect, administration route, term of treatment and the like. A satisfactory effect can be obtained by systemic administration 1-4 times per day or continuous administration at the amount of 0.00001-500 mg/kg per day, more preferably 0.0001-100 mg/kg.

The compound may preferably be formulated in pharmaceutical composition suitable for administration in a conventional manner. The composition may be those suitable for oral administration, intranasal administration, ocular topical administration, inhalational administration, injection or perfusion as well as it may be an external agent, suppository or pessary.

The composition of the present invention may further contain physiologically acceptable additives. Said additives may include the ingredients used with the present compounds such as excipient, diluent, filler, resolvent, lubricant, adjuvant, binder, disintegrator, coating agent, cupsulating agent, ointment base, suppository base, aerozoling agent, emulsifier, dispersing agent, suspending agent, thickener, tonicity agent, buffering agent, soothing agent, preservative, antioxidant, corrigent, flavor, colorant, a functional material such as cyclodextrin and biodegradable polymer, stabilizer. The additives are well known to the art and may be selected from those described in general reference books of pharmaceutics.

The amount of the above-defined compound in the composition of the invention may vary depending on the formulation of the composition, and may generally be 0.000001-10.0%, more preferably 0.00001-5.0%, most preferably 0.0001-1%.

Examples of solid compositions for oral administration include tablets, troches, sublingual tablets, capsules, pills, powders, granules and the like. The solid composition may be prepared by mixing one or more active ingredients with at least one inactive diluent. The composition may further contain additives other than the inactive diluents, for example, a lubricant, a disintegrator and a stabilizer. Tablets and pills may be coated with an enteric or gastroenteric film, if necessary. They may be covered with two or more layers. They may also be adsorbed to a sustained release material, or microcapsulated. Additionally, the compositions may be capsulated by means of an easily degradable material such gelatin. They may be further dissolved in an appropriate solvent such as fatty acid or its mono, di or triglyceride to be a soft capsule. Sublingual tablet may be used in need of fast-acting property.

Examples of liquid compositions for oral administration include emulsions, solutions, suspensions, syrups and elixirs and the like. Said composition may further contain a conventionally used inactive diluents e.g. purified water or ethyl alcohol. The composition may contain additives other than the inactive diluents such as adjuvant e.g. wetting agents and suspending agents, sweeteners, flavors, fragrance and preservatives.

The composition of the present invention may be in the form of spraying composition, which contains one or more active ingredients and may be prepared according to a known method.

Example of the intranasal preparations may be aqueous or oily solutions, suspensions or emulsions comprising one or more active ingredient. For the administration of an active ingredient by inhalation, the composition of the present invention may be in the form of suspension, solution or emulsion which can provide aerosol or in the form of powder suitable for dry powder inhalation. The composition for inhalational administration may further comprise a conventionally used propellant.

Examples of the injectable compositions of the present invention for parenteral administration include sterile aqueous or non-aqueous solutions, suspensions and emulsions. Diluents for the aqueous solution or suspension may include, for example, distilled water for injection, physiological saline and Ringer's solution.

Non-aqueous diluents for solution and suspension may include, for example, propylene glycol, polyethylene glycol, vegetable oils such as olive oil, alcohols such as ethanol and polysorbate. The composition may further comprise additives such as preservatives, wetting agents, emulsifying agents, dispersing agents and the like. They may be sterilized by filtration through, e.g. a bacteria-retaining filter, compounding with a sterilizer, or by means of gas or radioisotope irradiation sterilization. The injectable composition may also be provided as a sterilized powder composition to be dissolved in a sterilized solvent for injection before use.

The present external agent includes all the external preparations used in the fields of dermatology and otolaryngology, which includes ointment, cream, lotion and spray.

Another form of the present invention is suppository or pessary, which may be prepared by mixing active ingredients into a conventional base such as cacao butter that softens at body temperature, and nonionic surfactants having suitable softening temperatures may be used to improve absorbability.

In the present invention, the fatty acid derivative may be formulated into an ophthalmic composition and is topically administered to the eyes of the patient. The ophthalmic composition of the present invention includes any dosage form for ocular topical administration used in the field of ophthalmology, such as an ophthalmic solution, an eye drop and an eye ointment. The ophthalmic composition can be prepared in accordance with conventional means known in the relevant technical field.

According to the present invention, the tatty acid derivatives of the present invention are useful for modulating cytokine activity.

As used herein, the various forms of the term “modulate” are intended to include stimulation (e.g., increasing or upregulating a particular response or activity) and inhibition (e.g., decreasing or downregulating a particular response or activity).

As used herein, the term “cytokine” refers to any polypeptide or protein that affects the functions of cells and is a molecule which modulates interactions between cells in the immune, inflammatory, hematopoietic, neural, stress or wound healing response. Examples of cytokines include, but I not limited to, interleukin (IL) including over 30 type such as IL-1α, IL-1β, IL-2, -3, -4, -5, -6, -7, -8, -9, -10, -11 to -37; interferon (IFN) such as IFN-α, IFN-β and IFN-γ; tumor necrosis factor (TNF) such as TNF-α and TNF-β; transforming growth factor (TGF) such as TGF-α and TGF-β; colony stimulating factor (CSF) such as granulocyte-colony-stimulating factor (G-CSF), granulocyte-macrophage-colony-stimulating factor (GM-CSF), macrophage-colony Stimulating factor (M-CSF), erythropoietin (EPO), stem cell factor (SCF) and monocyte chemotactic and activating factor (MCAF); growth factor (GF) such as epidermal growth factor (EGF), fibroblast growth factor (FGF), insulin like growth factor (IGF), nerve growth factor (NGF), Brain-derived neurotrophic factor (BDNF), platelet derived growth factor (PDGF), vascular endothelial growth factor (VEGF), hepatocyte growth factor (HGF), keratinocyte growth factor (KGF), thrombopoietin (TPO), and bone morphogenic protein (BMP); and other polypeptide factors including LIF, kit ligand (KL), MPO (Myeloperoxidase) and CRP (C-reactive protein); COX (Cyclooxygenase) such as COX-1, COX-2 and COX-3, NOS (Nitric oxide synthase) such as NOS-1, NOS-2 and NOS-3; SOCS (suppressor of cytokine signaling) such as CIS, SOCS-1, -2, -3, -4, -5, -6 and -7; and so on.

Cytokines also includes chemokines which are cytokines that induce chemotaxis. There are two major classes of chemokines, CXC and CC. The CXC chemokines, such as neutrophil-activating protein-2 (NAP-2) and melanoma growth stimulatory activity protein (MGSA) are chemotactic primarily for neutrophils and T lymphocytes, whereas the CC chemokines, such as RANTES, Macrophage inflammatory protein (MIP) including MIP-1α and MIP-1β, keratinocyte-derived chemokine (KC), the monocyte chemotactic proteins (MCP-1, MCP-2, MCP-3, MCP-4, and MCP-5) and the eotaxins (-1 and -2) are chemotactic for, among other cell types, macrophages, T lymphocytes, eosinophils, neutrophils, dendritic cells, and basophils. There also exist the chemokines lymphotactin-1, lymphotactin-2 (both C chemokines), and fractalkine (a CX3C chemokine) that do not fail into either of the major chemokine subfamilies.

The fatty acid derivative of the present invention is especially useful for the modulation of IL-1β, IL-6, IL-12, TNF-α, IFN-γ, COX2, MPO, KC and SOCS-1.

The “cytokine activity” as used herein, includes cytokine-mediated signaling and expression of cytokine. “Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Proliferative activity” encompasses an activity that promotes, that is necessary for, or that is specifically associated with, e.g., normal cell division, as well as cancer, tumors, dysplasia, cell transformation, metastasis, and angiogenesis.

In one embodiment, the fatty acid derivatives of the present invention is useful for inhibiting expression of cytokines (e.g. gene or protein expression of IL-12, IL-1β, IL-6, TNF-α) in intestine or colon.

In one embodiment, the fatty acid derivatives of the present invention is useful for modulating expression of suppressor of cytokine's signaling (e.g. gene or protein expression of SOLS) in intestine or colon.

According to the present invention, the fatty acid derivatives, of the present invention are also useful for immunomodulation. Especially said immunomodulation is for the treatment of cytokine-mediated diseases such as autoimmune disease, neural disease, inflammatory disease, angiogenesis associated diseases including neoplasm.

As used herein, conditions with benefit from immunomodulation include, for example, but not limited to, Abortus habitualis, Achlorhydra autoimmune active chronic hepatitis, Acute disseminated encephalomyelitis (ADEM), Acute necrotizing hemorrhagic leukoencephalitis, Acute and chronic renal failure, Addison's disease, Adrenal insufficiency, Agammaglobulinemia, Allergic rhinitis, Allergic angiitis and granulomatosis, Alopecia greata, Amyloidosis, Alzheimer disease, Amyotrophic lateral sclerosis (ALS, Lou Gehrig's Disease), Angiogenesis, Ankylosing spondylitis, Anti-GBM Nephritis or anti-TBM Nephritis, Antiphospholipid syndrome (APS), Aplastic Anemia, Arthritis, Asthma, Atopic allergy, Atopic Dermatitis, Atherosclerosis, Aplastic anemia, Bullous pemphigoid, Cardiomyopathy, Chronic fatigue syndrome, Dermatomyositis, Dysautonomia, Epilepsy, Glomerulonephritis, Hemolytic anemia, Hepatitis, Hyperlipidemia, Immunodeficiency, Autoimmune inner ear disease (AIED), Autoimmune lymphoproliferative syndrome (ALPS), Myocarditis, Oophoritis, Pancreatitis, Autoimmune neutrogena, Pemphigus/Pemphigoid, Pernicious anemia, Polyarteritis nodosa, Polymyositis, Primary biliary cirrhosis, Retinopathy, Sarcoidosis, Autoimmune thrombocytopenic purpura (ATP), Thyroid disease, Ulcerative colitis, Uveitis, Vitiligo, Axonal & neuronal neuropathies, Balo Disease, Berger's Disease, Behchet's disease, Bullous Pemphigoid, Cardiomyopathy, Castleman disease, Celiac disease (Coeliac disease), Cerebellar degeneration, Chagas disease, Chronic asthmatic bronchitis, Chronic bronchitis, Chronic fatigue immune dysfunction syndrome (CFIDS), Chronic fatigue syndrome, Chronic inflammatory demyelinating polyneuropathy (CIDP), Chronic obstructive pulmonary disease (COPD), Chronic neuropathy with monoclonal gammopathy, Chronic recurrent multifocal ostomyelitis (CRMO), Churg Strauss syndrome, Cicatricial pemphigoid/Benign mucosal pemphigoid, Classic polyarteritis nodosa, Cogans syndrome, Cold agglutinin disease, Colitis, Congenital adrenal hyperplasia, Congenital heart block, Coxsackie myocarditis, Cranial Arteritis, CREST Syndrome, Cryopathies, Crohn's disease, Cushing's Syndrome, Dego's Disease, Demyelinating neuropathies, Dermatitis, Dermatitis herpetiformis, Dermatomyositis, Devic's disease (neuromyelitis optica), Diabetes, Type 1, Diabetes, Type 2, Discoid lupus, Dressler's syndrome, Eaton-Lambert myasthenic syndrome, Eczema, Emphysema, Endometriosis, Encephalomyelitis, Eosinophilic fasciitis, Epidermolysis Bullosa Acquisita, Erythema nodosa, Esophagitis or esophageal damage including esophageal ulcer, Essential mixed cryoglobulinemia, Evans syndrome, Experimental allergic encephalomyelitis, Fibromyalgia, Fibromyositis, Fibrosing alveolitis, Gastritis, Giant cell arteritis (temporal arteritis), Glomerulonephritis, Gluten-sensitive enteropathy, Goodpasture's syndrome, Grave's disease, Guillain-Barre syndrome, Hashimoto's disease (Hashimoto's thyroiditis,) Hepatitis C virus, Hemolytic anemia, Henoch-Schonlein purpura, Hepatitis, Herpes gestationis, Hidradenitis Suppurativa, Hughes Syndrome, HIV encephalopathia, Hyperthyroidism, Hypogammaglobulinemia, Idiopathic Adrenal Atrophy, Idiopathic hemachromatosis, Idiopathis membranous glomerulonephritis, Idiopathic pulmonary fibrosis, Idiopathic thrombocytopenic purpura (ITP), IgA nephropathy (IgA nephritis), IgG4-related sclerosing disease, Immunoregulatory lipoproteins, Inclusion body myositis, Inflammatory Demylinating Polyneuropathy, Interstitial cystitis, Irritable Bowel Syndrome, Isolated vasculitis of the central nervous system, Isaacs' syndrome, Juvenile arthritis, Juvenile diabetes, Kawasaki's disease, Lambert-Eaton syndrome, Leukocytoclastic vasculitis, Lichen planus, ichen sclerosus, Ligneous conjunctivitis, Linear IgA disease (LAD), Lou Gehrig's disease, Lupoid Hepatitis, Lupus erythematosus, systemic lupus erythematosus (SLE), Lupus nephritis, Lyme disease, Chronic lyme disease, Membranoproliferative glomerulonephriti, Meniere's disease, Microscopic polyangiitis, Minimal change renal disease, Miscellaneous vasculitides, Mixed connective tissue disease (MCTD), Mooren's ulcer, Morphea, Mucha-Habermann disease, Multifocal moter neuropathy with conduction block, Multiple myeloma, Multiple sclerosis, Myasthenia gravis, Myositis, Narcolepsy, Nephrotic syndrome, Neuromyelitis optica (Devic's), Neuromytonia, Neutropenia, Ocular cicatricial pemphigoid, Optic neuritis, Opsoclonus-myoclonus syndrome, Osteoporosis, Palindromic rheumatism, PANDAS (Pediatric Autoimmune Neuropsychiatric Disorders, Associated with Streptococcus), Paraneoplastic cerebellar degeneration, Paroxysmal nocturnal hemoglobinuria (PNH), Parkinson's disease, Parry Romberg syndrome, Pars planitis (peripheral uveitis), Parsonnage-Turner syndrome, Pediatrics autoimmune neuropsychiatry disorders, Pemphigoid, Pemphigus, Pemphigus Vulgaris, Peripheral neuropathy, Perivenous encephalomyelitis, Pernicious anemia, POEMS syndrome, Polyarteritis nodosa, Polyglandular Autoimmune Syndromes, Polymyalgia Rheumatica (PMR), Polymyositis, Post infective arthritides, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia, Pyoderma gangrenosum, Raynauds phenomenon, Reactive arthritides, Reflex sympathetic dystrophy, Reiter's syndrome, Relapsing polychondritis, Restless legs syndrome, RetinopaLhy, Retroperitoneal fibrosis, Rheumatic fever, Rheumatoid arthritis, Sarcoidosis, Schmidt syndrome, Sclerosing cholangitis, Scleritis, Scleroderma, Sjogren's syndrome, Sperm & testicular autoimmunity, Sticky Blood Syndrome, Stiff person syndrome, Still's Disease, Subacute thyroiditis, Subacute bacterial endocarditis (SBE), Susac's syndrome, Sydenham Chorea, Sympathetic ophthalmia, Synpharyngitic glomerulonephritis, Systemic Lupus Erythmatosis (SLE), Systemic necrotizing vasculitides, Systemic sclerosis, Takayasu's arteritis, Thromboangiitis obliterans, Thrombocytopenic purpura (TTP), Tolosa-Hunt syndrome, Transverse myelitis, Type I, II, & III autoimmune polyglandular syndromes, Ulcerative colitis, Undifferentiated connective tissue disease (UCTD), Uveitis, Vasculitis, Vesiculobullous dermatosis, Vitiligo, Wegener's granulomatosis, Wilson's disease, esophageal carcinoma, gastric carcinoma, duodenal cancer, small intestinal cancer, appendiceal cancer, large bowel cancer, colon cancer, rectum cancer, anal carcinoma, pancreatic cancer, liver cancer, gallbladder cancer, spleen cancer, renal carcinoma, bladder cancer, prostatic carcinoma, testicular carcinoma, uterine cancer, ovarian cancer, mammary carcinoma, pulmonary carcinoma and thyroid carcinoma.

Another embodiment of the present invention provides a treatment of esophagitis or esophageal damage.

The term “treating” or “treatment” used herein includes prophylactic and therapeutic treatment, and any means of control such as prevention, care, relief of the condition, attenuation of the condition, arrest of progression, etc.

The pharmaceutical composition of the present invention may contain a single active ingredient or a combination of two or more active ingredients, as far as they are not contrary to the objects of the present invention. For example, cytokines including chemokines, anti-body of cytokines such as anti TNF antibody (e.g. infliximab, adalimumab), anti-VEGF antibody (e.g. bevacizumab and ranibizumab), cytokine receptor antagonist such as anti HER2 antibody (e.g. Trastuzumab), anti EGF receptor antibody (e.g. Cetuximab), anti VEGF aptamer (e.g. Pegaptanib) and immunomodulator such as cyclosporine, tacrolimus, ubenimex may be used for the combination therapy.

In a combination of plural active ingredients, their respective contents may be suitably increased or decreased in consideration of their therapeutic effects and safety.

The term “combination” used herein means two or more active ingredient are administered to a patient simultaneously in the form of a single entity or dosage, or are both administered to a patient as separate entities either simultaneously or sequentially with no specific time limits, wherein such administration provides therapeutically effective levels of the two components in the body, preferably at the same time.

The present invention will be described in detail with reference to the following example, which, however, is not intended to limit the scope of the present invention.

### Example 1

Male LEW/SsN rats were given free access to 3% dextran sulfate sodium (DSS) in drinking water to induce ulcerative colitis. (−)-7-{(2R,4aR,5R,/aR)-2-[(3S)-1,1-difluoro-3-methylpentyl]-2-hydroxy-6-oxooctahydrocyclopenta[b]pyran-5-yl}heptanoic acid (Compound A) was orally administered to the animals twice a day for 10 days. The same amount of the vehicle was administered to control animals. At the 10 days after start of 3% DSS drinking, animals were sacrificed and intestinal tissues were excised. The mRNA expressions of IL-12, IL-1beta, IL-6, and TNF-alpha in the intestinal tissues were measured by real-time polymerase chain reaction technique.

In the control animals, the expressions of IL-12, IL-1beta, IL-6, and TNF-alpha were increased by the DSS drinking. Compound A reduced the expressions of IL-12, IL-1beta, IL-6, and TNF-alpha increased in DSS-induced ulcerative colitis model animals.

TABLE 1
Effects of Compound A on expression of cytokines

| Group | IL-12 | IL-1beta | IL-6 | TNF-alpha |
|---|---|---|---|---|
| Control | 1.69 | 90.49 | 93.88 | 7.57 |
| Compound A | 0.99 | 54.83 | 49.25 | 4.68 |

Each value is relative to the reference gene (GAPDH) level. Data are represented as mean (Arbitrary unit) from 3 to 4 animals.

The result indicated that the Compound of the present invention modulates the expressions of cytokines.

### Example 2

Patients who take NSAIDs were randomized to one of four treatment groups. All patients received 500 mg of naproxen twice a day. One group received placebo while the other three groups received 18, 36 or 54 mcg of Compound A, respectively, for 12 weeks. The incidence of esophagitis was 10.0%, 6.5% or 6.5% for the groups received 18, 36 or 54 mcg of Compound A, respectively, while it was 20.0% for the group received placebo.

The result indicates that the Compound of the present invention suppresses the incidence of esophagitis.

### Example 3

### Effects of Compound B on SOCS-1 Expression in DSS-Induced Colitis Model Mice

### Method

The experimental model used C57/B6J mice (7-8 weeks old). Colitis was induced by administration of 2% dextran sulfate sodium (DSS) in drinking water for 7 days. Ten (10) μM solution of Compound B ((−)-7-[(2R,4aR,5R,7aR)-2-(1,1-difluoropentyl)-2-hydroxy-6-oxooctahydrocyclopenta[b]pyran-5-yl]heptanoic acid) was administered orally once daily for 7 days from the day of starting DSS treatment.

One day after last administration, animals were sacrificed and colons were excised. After extraction of mRNA from colon tissues, real-time PCR analysis of SOCS-1 was conducted using house-keeping gene GAPDH as a reference.

### Results

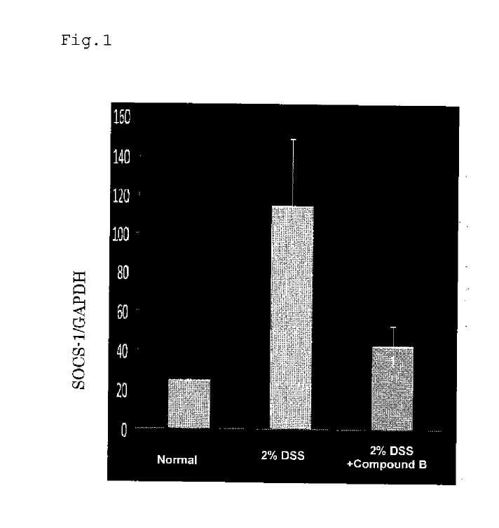

In 2% DSS treated animals, overexpression of SOCS-1 gene was observed compared to normal animals. This overexpression was reduced by Compound B treatment (see FIG. 1).

1. A method for modulating cytokine activity, immunomodulation or treating esophagitis in a mammalian subject, which comprises administering to the subject in need thereof an effective amount of a fatty acid derivative represented by the formula (I): 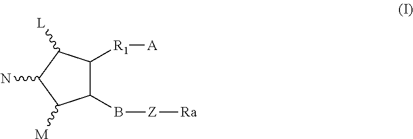
wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;
A is —CH₃, or —CH₂OH, —COCH₂OH, —COOH or a functional derivative thereof;
B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;
Z is 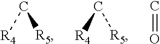
or single bond
wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;
R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

2. The method as described in claim 1, wherein Z is C═O.

3. The method as described in claim 1, wherein B is —CH₂—CH₂—.

4. The method as described in claim 1, wherein Z is C═O and B is —CH₂—CH₂—.

5. The method as described in claim 1, wherein L is hydroxy or oxo, M is hydrogen or hydroxy, N is hydrogen, B is —CH₂—CH₂— and Z is C═O.

6. The method as described in claim 1, wherein Ra is substituted by mono or dihalogen.

7. The method as described in claim 1, wherein B is —CH₂—CH₂—, Ra is substituted by mono or dihalogen.

8. The method as described in claim 1, wherein B is —CH₂—CH₂—, Z is C═O and Ra is substituted by mono or dihalogen.

9. The method as described in claim 1, wherein B is —CH₂—CH₂— and Ra is substituted by mono or difluoro.

10. The method as described in claim 1, wherein Z is C═O and Ra is substituted by mono or difluoro.

11. The method as described in claim 1, wherein B is —CH₂—CH₂—, Z is C═O and Ra is substituted by mono or difluoro.

12. The method as described in claim 1, wherein L is oxo, M is hydrogen or hydroxy, N is hydrogen, B is —CH₂—CH₂— and Ra is substituted by mono or dihalogen.

13. The method as described in claim 1, wherein L is oxo, M is hydrogen or hydroxy, N is hydrogen, Z is C═O, Ra is substituted by mono or dihalogen.

14. The method as described in claim 1, wherein L is oxo, M is hydrogen or hydroxy, N is hydrogen, B is —CH₂—CH₂—, Z is C═O and Ra is substituted by mono or dihalogen.

15. The method as described in claim 1, wherein L is oxo, M is hydrogen or hydroxy, N is hydrogen, B is —CH₂—CH₂—, R₁ is saturated bivalent lower of medium aliphatic hydrocarbon and Ra is substituted by mono- or difluoro.

16. The method as described in claim 1, wherein L is oxo, M is hydrogen or hydroxy, N is hydrogen, B is —CH₂—CH₂—, Z is C═O, R₁ is saturated bivalent lower of medium aliphatic hydrocarbon.

17. The method as described in claim 1, wherein said fatty acid derivative is (−)-7-[(2R,4aR,5R,7aR)-2-(1,1-Difluoropentyl)-2-hydroxy-6-oxooctahydrocyclopenta[b]pyran-5-yl]heptanoic acid, (−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid or (−)-7-[(1R,2R)-2-(4,4-difluoro-3-oxooctyl)-5-oxocyclopentyl]heptanoic acid or its functional derivative thereof.

18. The method as described in claim 1, said immunomodulation is for the treatment of cytokine-mediated diseases.
